(19) United States
(12) Patent Application Publication
(10) Pub. No.: US 2016/0025685 A1
(43) Pub. Date: Jan. 28, 2016

(54) PHANTOM FOR CALIBRATING OBJECT INFORMATION ACQUIRING APPARATUS AND MANUFACTURING METHOD THEREOF

(71) Applicant: CANON KABUSHIKI KAISHA, Tokyo (JP)

(72) Inventors: Takahiro Suita, Kyoto-shi (JP); Ryo Ogawa, Kawasaki-shi (JP); Yohei Hashizume, Nara-shi (JP); Yasufumi Asao, Kyoto-shi (JP)

(21) Appl. No.: 14/800,958

(22) Filed: Jul. 16, 2015

(30) Foreign Application Priority Data
Jul. 23, 2014 (JP) 2014-149767

(51) Int. Cl. G01N 29/30; A61B 5/00

(52) U.S. Cl. CPC G01N 29/30; A61B 5/0095; A61B 2560/0223

(57) ABSTRACT

A phantom used for calibrating an object information acquiring apparatus that irradiates light into an object and acquires characteristic information inside the object based on an acoustic wave propagated from the object, the phantom comprises a base material; and a simulated tissue portion that simulates a three-dimensional structure of a tissue inside a living body, wherein the simulated tissue portion is disposed inside the base material, and has a simulated layer, which is a layer simulating optical characteristics of the tissue inside the living body, on the surface thereof.

### BACKGROUND OF THE INVENTION

1. Field of the Invention

The present invention relates to the phantom simulating characteristics of a living body.

2. Description of the Related Art

Vigorous research on optical imaging apparatuses that generate images of information inside an object using the photoacoustic effect is on-going in medical fields.

If measurement light, such as pulsed laser light, is irradiated into a living body, which is an object, an acoustic wave is generated when the measurement light is absorbed by the biological tissue inside the object. By receiving and analyzing this acoustic wave (typically an ultrasound wave), information related to the optical characteristics inside the living body can be imaged. This acoustic wave is called a “photoacoustic wave”, and the technique using the photoacoustic wave is called “photoacoustic tomography (PAT)”.

A measurement apparatus for the photoacoustic tomography must be calibrated periodically to maintain measurement accuracy. Moreover, this measurement apparatus must maintain the operation status at shipment for safety, since laser light is irradiated. The apparatus is normally calibrated by measuring an artificial object (phantom) to be used as a reference, and adjusting the parameters based on the acquired images.

It is demanded that the phantom used for calibration of an apparatus simulates the light propagation characteristics and acoustic wave propagation characteristics of the living body (object). As a technique related to this, International Publication No. WO2013/077077, for example, disclosed a phantom where a pattern printed by ink simulating the characteristics of a tissue in the living body is disposed in the model of the tissue constituted by thermoplastic resin.

### SUMMARY OF THE INVENTION

The phantom disclosed in International Publication No. WO2013/077077 simulates the characteristics of the components constituting the living body by printing on a coating film made of hydrophilic resin, using ink having characteristics similar to the characteristics of biological tissue.

However, in the case of the method of simulating the characteristics of biological tissue using printing, it is very difficult to create a three-dimensional structure, such as a vascular structure. Hence reproducing clinical data on a blood vessel and a tumor structure very accurately, or predicting the image to be acquired, is not possible.

On the other hand, a technique to mold a micro three-dimensional structure using a 3D printer is lately attracting attention. However materials that can be used for a 3D printer are limited, and molding cannot be performed using a material of which optical characteristics, such as light absorbing properties and light scattering properties, have been adjusted.

With the foregoing in view, it is an object of the present invention to provide a phantom in which the structure and the optical characteristics inside the object are accurately simulated.

The present invention in its one aspect provides a phantom used for calibrating an object information acquiring apparatus that irradiates light into an object and acquires characteristic information inside the object based on an acoustic wave propagated from the object, the phantom comprises a base material; and a simulated tissue portion that simulates a three-dimensional structure of a tissue inside a living body, wherein the simulated tissue portion is disposed inside the base material, and has a simulated layer, which is a layer simulating optical characteristics of the tissue inside the living body, on the surface thereof.

The present invention in its another aspect provides a method of manufacturing a phantom used for calibrating an object information acquiring apparatus that irradiates light into an object and acquires characteristic information inside the object based on an acoustic wave propagated from the object, the method comprises a structure forming step of forming a simulated tissue portion, which is a structure simulating a three-dimensional structure of a tissue inside a living body; a simulated layer forming step of forming a simulated layer, which is a layer simulating optical characteristics of the tissue inside the living body, on the surface of the simulated tissue portion; and a base material forming step of forming a base material so as to include the simulated tissue portion on which the simulated layer is formed.

According to the present invention, a phantom in which the structure and optical characteristics inside the object are accurately simulated can be provided.

Further features of the present invention will become apparent from the following description of exemplary embodiments with reference to the attached drawings.

### BRIEF DESCRIPTION OF THE DRAWINGS

FIG. 1 is a diagram depicting a configuration of a phantom according to Embodiment 1;

FIG. 2 is a diagram depicting a configuration of a photoacoustic measurement apparatus according to Embodiment 1; and

FIG. 3 is a diagram depicting a configuration of a phantom according to Embodiment 2.

### DESCRIPTION OF THE EMBODIMENTS

Embodiments of the present invention will now be described with reference to the drawings. As a result, the same composing elements are denoted with same reference numerals, for which description is omitted. Numeric values, materials or the like, which are used for description of the embodiments, are not intended to limit the scope of the invention.

### Embodiment 1

A phantom according to Embodiment 1 is a phantom used for calibrating an object information acquiring apparatus using photoacoustic tomography (hereafter called “photoacoustic measurement apparatus”), and simulates the internal structure and characteristics of a living body, which is an object.

The photoacoustic measurement apparatus according to Embodiment 1 is an apparatus to image information related to the optical characteristics inside a living body by irradiating laser light into the living body, and receiving and analyzing a photoacoustic wave which is generated by the laser light inside the living body and is propagated. Normal information related to the optical characteristics includes: initial sound pressure distribution, light absorption energy density distribution, absorption coefficient distribution or density distribution of substances constituting the tissue.

The photoacoustic measurement apparatus must be calibrated periodically to maintain measurement accuracy. In concrete terms, a phantom for calibration is measured, and various parameters of the apparatus are adjusted while referring to the measurement result, so that information that should be acquired is acquired without deviation.

<Configuration of Phantom>

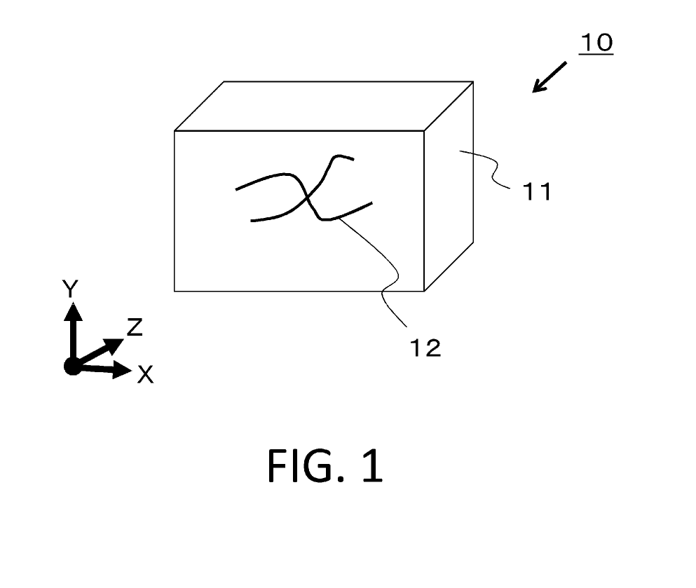

FIG. 1 is a diagram depicting a configuration of the phantom according to Embodiment 1. The phantom 10 in FIG. 1 is a phantom having a rectangular parallelepiped shape.

The reference numeral 11 denotes a phantom base material simulating an object tissue. The phantom base material is preferably constituted by a material simulating the acoustic characteristics and optical characteristics of the tissue inside the living body, such as a polyol or a polyol in which a compound having light scattering properties or light absorbing properties is dispersed. For the polyol, it is preferable to use polyether polyol because of the correlation in terms of the acoustic characteristics of the tissue in the living body.

The reference numeral 12 denotes a simulated tissue portion in the present invention, and is a structure (simulated blood vessels) molded by simulating the shape of a tissue inside the object. In this embodiment, a three-dimensional structure of blood vessels in the living body is molded as simulated blood vessels using a 3D printer. The material used for the molding is preferably acrylic resin, which has high transmittance in a near infrared region, and can be used by a 3D printer.

The simulated blood vessels 12 are generated by reproducing the shape of the tissue inside the living body, but the resin that can be used by the 3D printer has high light transmittance, and does not generate acoustic waves very much, hence it cannot be directly used as a material of a phantom. Therefore in this embodiment, a layer simulating the optical characteristics of the tissue in the living body (blood vessels in this embodiment) is formed on the surface of the simulated blood vessels 12. In concrete terms, a polyol layer simulating the optical characteristics in a near infrared region is formed on the portion corresponding to the arteries and the portion corresponding to the veins respectively. Hereafter this layer is called “simulated layer”.

To approximate the light propagating characteristics of the base material 11 and the simulated layer to the light propagating characteristics of the tissue in the living body, the equivalent scattering coefficient and absorption coefficient must be adjusted by dispersing a compound having light scattering properties or absorbing properties.

In this embodiment, a polyether dispersing agent having an affinity with carbon black pigment is used as the compound having light absorbing properties. Needless to say, other pigments or dispersing agents may be used.

To disperse a compound in polyol, it is necessary to perform surface modification or the like on the particles in order to enhance dispersion uniformity. If titanium oxide is used as a compound having the light scattering properties, performing surface treatment using aluminum oxide and hexamethyldisilazane is preferable. Needless to say, titanium oxide treated by other surface treatment methods may be used.

The light propagation characteristics of the simulated layer will be described in detail.

The simulated layer of this embodiment contains two types of compounds, of which absorption coefficient ratios in arbitrary two wavelengths (λ₁, λ₂, λ₁<λ₂) in the 600 nm to 1100 nm range, are different from each other. These compounds are dispersed at an arbitrary ratio, so that the absorption coefficient ratio of the arbitrary two wavelengths can be reproduced.

Here if the absorption coefficient at each wavelength is set as follows, the oxygen saturation S can be determined by Expression 1.

$$\begin{matrix} [ Math . 1 ] & \\ s = ( μ [ {λ}_{2} ] / μ [ {λ}_{1} ] ) \cdot Hb [ {λ}_{1} ] - Hb [ {λ}_{2} ] ( {Hb O}_{2} [ {λ}_{2} ] - Hb [ {λ}_{2} ] ) - ( \frac{μ [ {λ}_{2} ]}{μ [ {λ}_{1} ]} ) \cdot ( {Hb O}_{2} [ {λ}_{1} ] - Hb [ {λ}_{1} ] ) \cdot 100 & ( Expression 1 )\end{matrix}$$

where

HbO₂[λ₁]: absorption coefficient of oxyhemoglobin at wavelength λ₁
HbO₂[λ₂]: absorption coefficient of oxyhemoglobin at wavelength λ₂
Hb [λ₁]: absorption coefficient of deoxyhemoglobin at wavelength λ₁
Hb [λ₂]: absorption coefficient of deoxyhemoglobin at wavelength λ₂
μ [λ₁]: absorption coefficient of blood vessel model at wavelength λ₁
μ [λ₂]: absorption coefficient of blood vessel model at wavelength λ₂

Now a method for calculating the absorption coefficient of the blood vessel model is shown below. It is assumed that the absorption coefficients of the compounds A and B at wavelengths λ₁ and λ₂ are μ_{A}[λ₁], μ_{B}[λ₁], μ_{A}[λ₂], μ_{B} [λ₂] respectively. If the concentration of the compounds A and B in the blood vessel model are C_{A} and C_{B}, then the following relationship is established.

μ[λ₁]=C_{A}·μ_{A}[λ₁]+C_{B}·μ_{B}[λ₁]

μ[λ₂]=C_{A}·μ_{A}[λ₂]+C_{B}·μ_{B}[λ₂]

If the absorption coefficient ratios of the compounds A and B, μ_{A}[λ₂]/μ_{A}[λ₁] and μ_{B}[λ₂]/μ_{B}[λ₁], are constant, then μ[λ₂]/μ[λ₁] becomes constant and oxygen saturation S cannot be controlled. In other words, by changing the concentration of the compounds A and B of which absorption coefficient ratios are different, the absorption coefficient ratio μ_{A}[λ₂]/μ_{A}[λ₁] and the oxygen saturation of the blood vessel model can be set to arbitrary values.

In this embodiment, the blood vessel model simulating the oxygen saturation of the arteries and veins respectively is created by adjusting the dispersion amount of the phthalocyanine compound, of which absorption coefficient ratios at two wavelengths (756 nm (λ₁) and 797 nm (λ₂)) are different.

In this embodiment, copper phthalocyanine (maximum absorption wavelength: 825 nm) and phthalocyanine vanadium complex (maximum absorption wavelength: 750 nm) are used as the phthalocyanine compound of which absorption ratios are different at each wavelength, but other compounds may be used.

In this embodiment, two types of compounds, of which absorption coefficient ratios are different, are used, but three or more types may be combined.

If measurement light is irradiated into the phantom configured like this, and a generated photoacoustic wave is received and imaged, the difference of the absorption coefficients of the phantom base material and the simulated layer appears as brightness. Further, the oxygen saturation can be computed using the absorption coefficient acquired at each wavelength. Hereafter an image acquired by measuring the phantom 10 according to this embodiment is called a “phantom image”.

<Method of Manufacturing Phantom>

A method of manufacturing the phantom 10 according to Embodiment 1 will be described next.

First the materials used for the phantom base material 11 are prepared. The phantom base material 11 of this embodiment is generated by dispersing a compound having light scattering properties or light absorption properties in the polyol contained in a beaker, stirring and performing vacuum defoaming. In this embodiment, titanium oxide (average particle diameter: 0.21 μm), which is surface treated by aluminum oxide and hexamethyldisilazane, is used as a compound having the light scattering properties. As the compound having the light absorbing properties, a mixture of carbon black pigment and polyether dispersing agent, which has an affinity with the pigment, is dispersed. For polyol, polyether polyol copolymer (number of average molecular weight: 6000), of which mol ratio of ethylene oxide and propylene oxide is 1:1, is used.

Then the simulated blood vessels 12 are formed (structure forming processing). In this embodiment, the blood vessel structure having a 50 μm to 2 mm diameter was molded using a 3D printer (Objet Eden 350V, made by Stratasys Ltd.) as the simulated blood vessels 12. The material appropriate for the molding is acrylic resin, of which transmittance in the near infrared region is 98%, and which can be used by the 3D printer.

Then the simulated layer is formed on the surface of the simulated blood vessels 12 (simulated layer forming processing).

In this embodiment, a polyurethane layer, of which thickness is 0.2 mm, is formed on the surface of the simulated blood vessels 12 by coating and thermally curing HDI added polyol, which has been adjusted so that the optical characteristics thereof becomes the same as those of the blood vessels (arteries or veins), on the surface of the simulated blood vessels 12. By forming the simulated layer using polyurethane having acoustic characteristics similar to the base material, the generation of an artifact is suppressed when the simulated blood vessels 12 are measured by the photoacoustic measurement apparatus.

The material used here need not be polyurethane. For example, thermoplastic polyurethane resin, acrylic resin, epoxy resin or the like may be used.

The simulated layer may be formed in the shape of the simulated blood vessels 12 using such methods as brush painting, spray coating and dip coating, and the forming method thereof is not especially limited.

Simulated blood vessels 12 after the simulated layer is formed are disposed so as to be included in the phantom base material 11 using a transparent nylon wire (diameter: 0.13 mm), which does not generate an acoustic wave when light is irradiated. Then hexamethylene diisocyanate (HDI), which is a curing agent, is added to the polyol, is stirred, vacuum defoaming is performed, then polyol is poured into a predetermined die and cured by heat (base material forming processing).

In the phantom in this embodiment, the acoustic characteristics are adjusted by an addition amount of HDI, the light absorption characteristics are adjusted by a dispersion amount of carbon black pigment, and the equivalent scattering coefficient is adjusted by a dispersion amount of titanium oxide.

<Configuration of Photoacoustic Measurement Apparatus>

The photoacoustic measurement apparatus that performs calibration using the above mentioned phantom will be described next. The targets that the photoacoustic measurement apparatus measures are a phantom, a living body or the like, and here all are collectively referred to as an “object”.

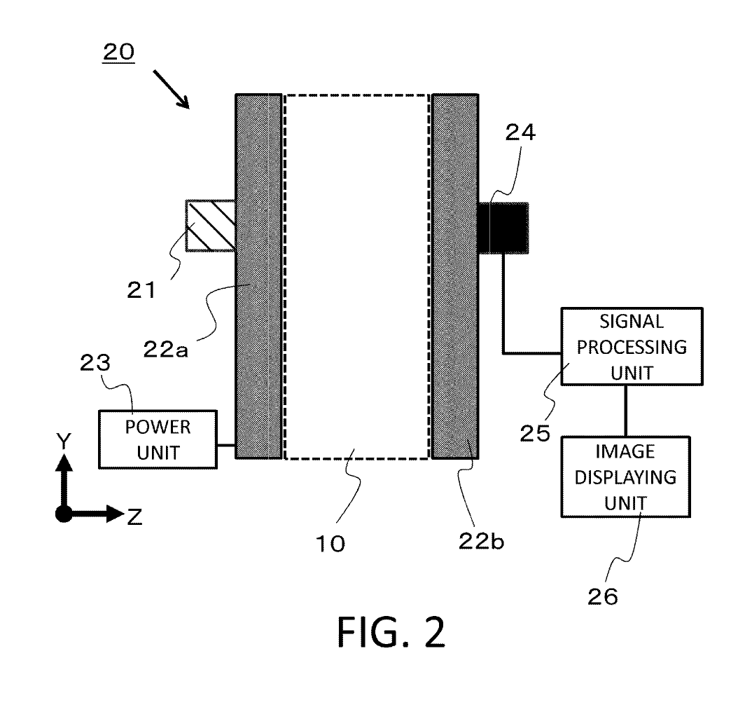

FIG. 2 is a system block diagram depicting the photoacoustic measurement apparatus 20 according to Embodiment 1.

The photoacoustic measurement apparatus 20 according to this embodiment includes: a light source 21 holding plates 22a and 22b, a power unit 23, an acoustic wave probe 24, a signal processing unit 25, and an image displaying unit 26. In this embodiment, a phantom 10 is held between the holding plates 22a and 22b.

<Light Source 21>

The light source 21 is an apparatus for generating pulsed light that is irradiated into an object. The light source is preferably a laser light source for acquiring high power, but a light emitting diode, a flash lamp or the like may be used instead of a laser. If a laser is used for the light source, various types of lasers can be used, such as a solid-state laser, a gas laser, a dye laser and a semiconductor laser. The irradiation timing, waveform, intensity or the like are controlled by a light source control unit, which is not illustrated. This light source control unit may be integrated with the light source.

The wavelength of the pulsed light is a specific wavelength that is absorbed by a specific component out of the components constituting the object, and preferably is a wavelength that allows the light to propagate inside the object. In concrete terms, if the object is a living body, a wavelength in the 650 nm to 1100 nm range, where the absorption spectrum of hemoglobin in blood (oxyhemoglobin and deoxyhemoglobin) has a distinctive feature, is preferable.

To effectively generate the photoacoustic wave, light must be irradiated within a sufficiently short time in accordance with the thermal characteristics of the object. If the object is a living body, the pulse width of pulsed light generated from the light source is preferably 10 to 50 nanoseconds.

In this embodiment, a titanium sapphire (Ti—S) laser-based light source that can emit two wavelengths, 756 nm and 797 nm, is used.

The pulsed light generated from the light source 21 is hereafter called “measurement light”.

Although omitted in FIG. 1, the light source 21 may be configured such that the measurement light emitted from the light source 21 is guided to the object via such optical components as a lens, a mirror, a diffusion plate and an optical fiber. For example, the measurement light may be expanded using an optical lens, so that the entire area of the contact surface of the holding plate 22a and the object is irradiated.

The light emitted from the light source 21 is irradiated onto the surface of the object via the later mentioned holding plate.

<Holding Plates 22a and 22b>

The holding plates 22a and 22b are units to hold the object (phantom 10). In concrete terms, the object is compressed and held by one or both of the two plate-shaped holding member(s) that move in horizontal directions of FIG. 2.

The measurement light emitted from the light source 21 is irradiated onto the surface of the object via the holding plate 22a, hence the holding plate 22a is preferably constituted by a material which has high transmittance with respect to the measurement light and a low attenuation rate. Typically glass, polymethyl pentene, polycarbonate, acryl or the like is preferable.

The acoustic wave generated in the object enters the acoustic wave probe 24 via the holding plate 22b. Hence the holding plate 22b is preferably constituted by a material which does not reflect the acoustic wave on the interface of the object, and easily transmits the acoustic wave. In concrete terms, a material of which difference of acoustic impedance from the object is small, is preferable. Such a material is a resin material, such as polymethyl pentene.

Further, it is preferable that the holding plates 22 have a thickness to maintain a certain strength so as to avoid deformation caused by the holding pressure, and typically about a 10 mm thickness is preferable. The holding plates 22 can be any size if the object can be held, but a size similar to that of the object is preferable.

In this embodiment, polymethyl pentene is used for the holding plates 22a and 22b.

If the phantom 10 is disposed between the holding plates 22a and 22b, foaming between the phantom and holding plates may be prevented by coating water or gel on the surface of the phantom.

<Power Source 23>

The power source 23 is a unit to move at least one of the holding plates 22a and 22b. The power source 23 may be a DC motor, an AC motor, a stepping motor or a linear actuator.

<Acoustic Wave Probe 24>

The acoustic wave probe 24 is a unit to receive an acoustic wave generated by the measurement light irradiated into the object, and convert the acoustic wave into an analog electric signal. The acoustic wave in this invention includes an elastic wave called a sound wave, ultrasound wave, photoacoustic wave and light induced ultrasound wave, and typically is an ultrasound wave. The acoustic wave probe 24 is also called a “probe” or “transducer”. The acoustic wave probe 24 may be constituted by a single acoustic detector or a plurality of acoustic detectors.

The acoustic wave probe 24 may have a plurality of light receiving elements that are one-dimensionally or two-dimensionally disposed. If multi-dimensionally arrayed elements are used, an acoustic wave can be simultaneously received at a plurality of locations, whereby the measurement time can be decreased. If the probe is smaller than the object, the acoustic wave probe may be scanned so that an acoustic wave is received at a plurality of locations.

It is preferable that the acoustic wave probe 24 has high sensitivity and a wide frequency band. In concrete terms, PZT (piezoelectric ceramic), PVDF (polyvinylidene fluoride resin), CMUT (capacitive micro-machine ultrasonic transducer), Fabry-Perot interferometer or the like may be used. The acoustic wave probe 24 is not limited to these examples, but can be anything as long as the functions of the probe are implemented.

In this embodiment, a probe of which oscillator is constituted by lead zirconate titanate (PZT) is used as the acoustic wave probe 24. The pitch of the oscillator is 1 mm and the central frequency thereof is 1 MHz.

<Signal Processing Unit 25>

The signal processing unit 25 is a unit to amplify the electric signal acquired by the acoustic wave probe 24, and convert the electric signal into a digital signal. Typically the signal processing unit 25 is constituted by an amplifier, an A/D converter, a FPGA (Field Programmable Gate Array) chip and the like. If a plurality of detection signals is acquired by the probe, it is preferable that the plurality of signals can be processed simultaneously. The amplifier may be embedded in the acoustic wave probe 24.

The signal processing unit 25 is also a unit to generate (reconstruct) an image by processing digital signals. Any method can be used for the image reconstruction method, such as a Fourier transform method, universal back projection method, filtered back projection method and iterative reconstruction method.

The signal processing unit 25 has a function to perform calibration by measuring the phantom. In concrete terms, the light absorption coefficient, equivalent scattering coefficient, sound speed and resolution to reconstruct an image or the like of the phantom in the initial state are stored in the memory as true values. The signal processing unit 25 also has a function to compare the true value and the measured value, and correct measurement errors. If the true value and measured value are significantly different, the signal processing unit 25 can perform error determination.

The signal processing unit 25 may be a computer that includes a CPU, a main storage apparatus and an auxiliary storage apparatus, or may be dedicated hardware.

<Image Displaying Unit 26>

The image displaying unit 26 is a unit to display a reconstructed image generated by the signal processing unit 25. For the image displaying unit 27, a liquid crystal display, a plasma display, an organic EL display, an FED or the like can be used.

<Method of Measuring Object>

A method of measuring a living body (object) by the photoacoustic measurement apparatus according to this embodiment will be described next.

First the living body (breast in this embodiment, which is not illustrated) is compressed and held using the holding plates 22. In this embodiment, the acoustic wave probe 24 is parallel with the measurement light irradiation direction in the Y axis direction with respect to the phantom 10.

Then the pulsed light emitted from the light source 21 is irradiated into the living body. If a part of the energy of the light propagated inside the living body is absorbed by a light absorber, such as blood, an acoustic wave is generated from the light absorber by thermal expansion. If cancer exists in the living body, the light is uniquely absorbed by the new blood vessels of the cancer in the same manner as the blood in other healthy areas, and an acoustic wave is generated. The photoacoustic wave generated in the living body is received by the acoustic wave probe 24.

The signal received by the acoustic wave probe 24 is analyzed by the signal processing unit 25. The analysis result is converted into image data (phantom image) that represents the characteristic information inside the living body (e.g. initial sound pressure distribution and absorption coefficient distribution), and is outputted through the image displaying unit 26. The phantom image may be, for example, three-dimensional data or may be two-dimensional data. To calibrate the apparatus, various parameters of the apparatus are adjusted based on the generated phantom image (or data that represents the characteristic information inside the object).

The concrete physical property values in this embodiment will be described. In this embodiment, the acoustic characteristics of the phantom base material 11 are: sound speed 1388 m/s, density 1.03, acoustic impedance 1.43 rayl, and acoustic attenuation 0.35 dB/cm/MHz at 37° C. The optical characteristics of the phantom base material 11 are: the absorption coefficient 0.004/mm and the equivalent scattering coefficient 0.93/mm at wavelength 797 nm. These light propagation characteristics and acoustic propagation characteristics are approximately the same as the soft tissue of the living body.

The acoustic characteristics of the simulated blood vessels 12 are: sound speed 1792 m/s, density 1.12, acoustic impedance 2.01 rayl, and acoustic attenuation 11.5 dB/cm/MHz at 37° C. The acoustic impedance of the simulated blood vessels 12 is greater than that of the phantom base material, but an artifact that may cause calibration problems is not generated since the structure of the simulated blood vessels is very small, and a resin layer close to the soft tissue of the living body is disposed on the surface of the material.

The optical characteristics of the portion simulating the arteries, out of the simulated blood vessels 12 after the simulated layer is formed, are the absorption coefficient 0.188/mm at wavelength 756 nm, and absorption coefficient 0.254/mm at wavelength 797 nm. In other words, the oxygen saturation calculated from the absorption coefficient ratio of the two wavelengths is 97.3%.

The optical characteristics of the portion simulating the veins, out of the simulated blood vessels 12 after the simulated layer is formed, are the absorption coefficient 0.257/mm at wavelength 756 nm, and absorption coefficient 0.255/mm at wavelength 797 nm. In other words, the oxygen saturation calculated from the absorption coefficient ratio of the two wavelengths is 75.9%.

As described above, according to Embodiment 1, a phantom that simulates a three-dimensional blood vessel structure in the living body, and simulates the photoacoustic wave generated from the blood vessels can be provided. Furthermore, a highly accurate measurement image used for calibration of the apparatus can be generated by measuring the phantom by the photoacoustic measurement apparatus.

### Embodiment 2

In Embodiment 1, the simulated blood vessels molded using a 3D printer become the simulated tissue portion. In Embodiment 2, on the other hand, a tumor is molded using a 3D printer, and the tumor in which the simulated blood vessels are disposed becomes the simulated tissue portion.

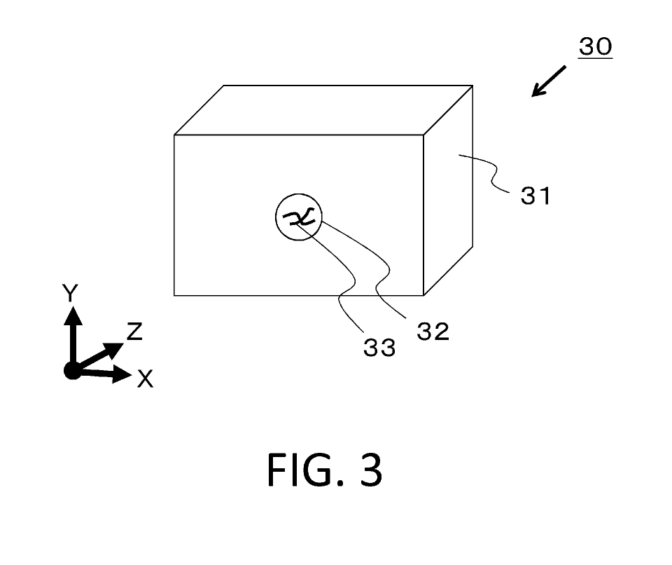

FIG. 3 is a diagram depicting a configuration of a phantom 30 according to Embodiment 2. In this embodiment 2, a member simulating the blood vessel structure inside the tumor (simulated blood vessels 33) is disposed inside a member simulating the tumor (simulated tumor 32), and this simulated tumor 32 is disposed inside a phantom base material 31.

In Embodiment 2, the simulated blood vessels 33 having a diameter of about 50 μm to 100 μm are formed using the 3D printer. As the simulated layer, a polyol layer, simulating the optical characteristics in a near infrared region of the veins, is formed on the surface of the simulated blood vessels 33. The simulated tumor 32 is a transparent sphere of which diameter is 5 mm, is formed using a same material as the base material, and contains the simulated blood vessels. The simulated tumor 32 is disposed inside the phantom base material 31 using a transparent nylon wire (diameter: 0.13 mm), which does not generate an acoustic wave when light is irradiated.

Unlike the simulated blood vessels 33, the simulated layer for simulating the optical characteristics inside the living body is not disposed on the simulated tumor 32.

The optical characteristics of the simulated blood vessels 33 contained in the simulated tumor 32 are the absorption coefficient 0.033/mm at wavelength 756 nm, and the absorption coefficient 0.026/mm at wavelength 797 nm. In other words, the oxygen saturation, calculated from the absorption coefficient ratio of the two wavelengths, is 54.8%.

Generally a malignant tumor in a living body requires oxygen, and the oxygen saturation thereof is lower than peripheral areas. In the phantom according to Embodiment 2, the layer simulating the optical characteristics in the veins is used as the simulated layer, whereby the oxygen saturation inside the tumor can be simulated.

As described above, the phantom according to Embodiment 2 can simulate the tumor segment in the living body by containing the simulated blood vessels in the simulated tumor. The acoustic wave generated in the blood vessels inside the tumor attenuates due to the presence of the tumor, and in Embodiment 2, even attenuation of the acoustic wave can be simulated since of the simulated tumor is disposed. In other words, the photoacoustic wave generated from the tumor segment (specifically the blood vessels in the tumor) can be accurately simulated. Furthermore, a highly accurate measurement image for calibration of the apparatus can be generated by measuring the phantom by the photoacoustic measurement apparatus.

(Modifications)

Each embodiment described above is an example, and the present invention can be carried out by appropriately modifying or combining within a scope not departing from the true spirit of the invention.

For example, the present invention may be carried out as an object information acquiring apparatus that includes at least a part of the above mentioned processings. Further, the present invention may be carried out as an object information acquiring system that includes the exemplified phantom and the object information acquiring apparatus. The processing and units described above, may be freely combined as long as no technical inconsistency is generated.

In the description on the embodiments, a copolymer of ethylene oxide and propylene oxide, of which mol ratio is 1:1 and number average molecular weight is 6000, is used, but another polyol may be used. In the description on the embodiments, polyol resin is cured using hexamethylene diisocyanate (HDI), but other isocyanate compounds and curing agents may be used.

In the description on the embodiments, the simulated tissue portion is formed by molding resin using the 3D printer, but the simulated tissue portion may be formed by other methods.

### Other Embodiments

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2014-149767, filed on Jul. 23, 2014, which is hereby incorporated by reference herein in its entirety.

1. A phantom used for calibrating an object information acquiring apparatus that irradiates light into an object and acquires characteristic information inside the object based on an acoustic wave propagated from the object, the phantom comprising:
a base material; and
a simulated tissue portion that simulates a three-dimensional structure of a tissue inside a living body, wherein
the simulated tissue portion is disposed inside the base material, and has a simulated layer, which is a layer simulating optical characteristics of the tissue inside the living body, on the surface thereof.

2. The phantom according to claim 1, wherein the base material is constituted by a material simulating acoustic characteristics inside the living body.

3. The phantom according to claim 1, wherein the simulated tissue portion has a three-dimensional structure simulating blood vessels inside the living body.

4. The phantom according to claim 3, wherein the simulated layer is a layer simulating optical characteristics of arteries or veins.

5. The phantom according to claim 3, wherein the simulated layer is a layer simulating optical characteristics with respect to an oxygen saturation of hemoglobin in the blood.

6. The phantom according to claim 3, further comprising a second simulated tissue portion that has a three-dimensional structure simulating a tumor in the living body,
wherein the simulated tissue portion is disposed inside the second simulated tissue portion.

7. A method of manufacturing a phantom used for calibrating an object information acquiring apparatus that irradiates light into an object and acquires characteristic information inside the object based on an acoustic wave propagated from the object, the method comprising:
a structure forming step of forming a simulated tissue portion, which is a structure simulating a three-dimensional structure of a tissue inside a living body;
a simulated layer forming step of forming a simulated layer, which is a layer simulating optical characteristics of the tissue inside the living body, on the surface of the simulated tissue portion; and
a base material forming step of forming a base material so as to include the simulated tissue portion on which the simulated layer is formed.

8. The method of manufacturing a phantom according to claim 7, wherein in the base material forming step, the base material is formed using a material simulating acoustic characteristics inside the living body.

9. The method of manufacturing a phantom according to claim 7, wherein in the structure forming step, a three-dimensional structure simulating blood vessels inside the living body is formed as the simulated tissue portion.

10. The method of manufacturing a phantom according to claim 9, wherein in the simulated layer forming step, a layer simulating optical characteristics of arteries or veins is formed as the simulated layer.

11. The method of manufacturing a phantom according to claim 9, wherein in the simulated layer forming step, a layer simulating optical characteristics with respect to an oxygen saturation of hemoglobin in the blood is formed as the simulated layer.

12. The method of manufacturing a phantom according to claim 9, further comprising:
a second structure forming step of forming a second simulated tissue portion, which is a structure simulating a tumor in the living body; and
a disposing step of disposing the simulated tissue portion inside the second simulated tissue portion.
